(19) United States
(12) Patent Application Publication
(10) Pub. No.: US 2025/0164457 A1
(43) Pub. Date: May 22, 2025

(54) METHOD FOR DETERMINING CARBON ISOTOPE COMPOSITION OF LIGHT HYDROCARBON MONOMERS IN SEMI-CLOSED PYROLYSIS SYSTEM

(71) Applicant: NORTHWEST INSTITUTE OF ECO-ENVIRONMENT AND RESOURCES, CAS, Lanzhou (CN)

(72) Inventors: Huifei TAO, Lanzhou (CN); Dongzheng MA, Lanzhou (CN); Zhen QIN, Lanzhou (CN); Chengfu LV, Lanzhou (CN); Zhongping LI, Lanzhou (CN); Long SU, Lanzhou (CN)

(21) Appl. No.: 19/034,053

(22) Filed: Jan. 22, 2025

(30) Foreign Application Priority Data
Dec. 13, 2024 (CN) 202411834693.4

(51) Int. Cl. G01N 33/22; G01N 1/42; G01N 1/44; G01N 30/72

(52) U.S. Cl. CPC G01N 33/225; G01N 1/42; G01N 1/44; G01N 30/7206

(57) ABSTRACT

A semi-closed pyrolysis system, including a liquid nitrogen freezing device, a gas-liquid collector and a pyrolysis gas collecting device. The liquid nitrogen freezing device includes a collecting vessel, a liquid nitrogen storage, an air inlet pipe, an air outlet pipe and a temperature detector. The collecting vessel is arranged in the liquid nitrogen storage container, and is provided with a stainless steel cap. The stainless steel cap is provided with three holes respectively for the air inlet pipe, the air outlet pipe and the temperature detector to pass through. The air inlet pipe and the air outlet pipe are each provided with a valve. The temperature detector includes a probe and a temperature sensor electrically connected with the probe. The probe extends into the collecting vessel. A method for determining carbon isotope composition of light hydrocarbon monomers based on such system is further provided.

### CROSS-REFERENCE TO RELATED APPLICATIONS

This application claims the benefit of priority from Chinese Patent Application No. 202411834693.4, filed on Dec. 13, 2024. The content of the aforementioned application, including any intervening amendments thereto, is incorporated herein by reference in its entirety.

### TECHNICAL FIELD

This application relates to oil and gas geology, and more particularly to a method for determining a carbon isotope composition of light hydrocarbon monomers in a semi-closed pyrolysis system.

### BACKGROUND

The geochemical characteristics of light hydrocarbons (C₅⁺) play an important role in natural gas exploration. However, it is difficult to enrich and analyze C₅⁺ light hydrocarbons.

The current improvements in the prior art focus on the enrichment and analysis of C₅⁺ hydrocarbon compounds. Natural gas is obtained from places with proven reserves. However, when research results are characterized and possible reserves nearby or in surrounding areas are predicted based on information about proven reserves of the natural gas in the same basin, there are often errors because lateral migration is a crucial and complex step in the process of oil and gas accumulation. When hydrocarbons migrate laterally, composition and isotopes of C₅⁺ vary greatly, and a distance between a discovered oil and gas storage site and a possible new reserve may be tens of kilometers. Therefore, besides of analysis of the proven reserves of natural gas, the researchers will also utilize potential hydrocarbon source rocks in different layers in the basin to conduct pyrolysis experiments.

The pyrolysis experiments can be divided into three types according to closure, which are an open system pyrolysis experiment, a closed system pyrolysis experiment and a semi-open (semi-closed) system pyrolysis experiment.

In the closed system pyrolysis experiment, a gold tube is an important experimental vessel. The gold tube utilizes chemical inertness and plasticity of gold, and is configured as a vessel for pyrolysis reaction of samples containing organic matters to generate hydrocarbons.

In the prior art, the gold tube is usually immersed in an organic solution dichloromethane. A vessel containing the dichloromethane is placed in liquid nitrogen, and the gold tube is pierced in the dichloromethane, and hydrocarbon compounds are dissolved in the dichloromethane to complete collection of all liquid hydrocarbons including light hydrocarbons (including C₁-C₁₃ hydrocarbons). Because of a certain volume of the gold tube, an amount of required dichloromethane is more, typically exceeding 50 ml per single use. Dichloromethane is classified as a 2A carcinogen, which is not user-friendly for experimental operators. Furthermore, once pierced, the gold tube cannot be reused and requires reprocessing for utilization. Therefore, the method herein is time-consuming and laborious.

However, by using the gold tube pyrolysis method to prepare light hydrocarbons, a small amount of crude oil and heavy hydrocarbon gas in the closed system failed to be discharged and cracked to form a gas with low molecular weight (mainly methane). Therefore, besides of the light hydrocarbons in the pyrolysis gas, a large amount of pyrolysis liquid and residual hydrocarbons in internal pores of a solid residue are mixed in the gold tube, which is quite different from information in an actual exploration.

It would be ideal to only collect the light hydrocarbons in the pyrolysis gas for comparison with light hydrocarbons in the natural gas, that is, gas components in the pyrolysis gas is compared to that in the natural gas rather than oil components in the in the pyrolysis gas is compared to gas components in the natural gas.

The above experiment is followed by rapid transfer of dichloromethane dissolved with the hydrocarbons into a glass bottle. Then the dichloromethane dissolved with the hydrocarbons is injected into an injection port of gas chromatography (GC) for gas chromatography analysis.

An injection volume of such hydrocarbon compounds mixed with C₁-C₄ hydrocarbons and C₅⁺ hydrocarbons is difficult to be determined when such hydrocarbon compounds is directly subjected to gas chromatography analysis. Considering that the content of C₁-C₄ hydrocarbons is generally more than 2 orders of magnitude higher than that of C₅⁺ hydrocarbons, the injection with a mixture of C₁-C₄ hydrocarbons and C₅⁺ hydrocarbons may raise the following problems.

(1) In the case of excessive injection, although C₅⁺ hydrocarbons that is eluted later exhibit a good peak pattern, peak coalescence may occur for C₁-C₄ hydrocarbons that are eluted earlier.

(2) In the case of insufficient injection, the peak overlap is not observed, but many C₅⁺ hydrocarbon compounds may not be detected because of low contents.

Therefore, it is necessary to separate C₁-C₄ hydrocarbons from C₅⁺ hydrocarbons in the hydrocarbon mixtures.

A semi-closed system hydrocarbon generation simulation experiment system is often used to simulate generation and discharge processes of hydrocarbons from source rocks in deep basin under high temperature and high pressure. Such system has advantages of large sample loading volume, many pyrolysis products, flexible application of geological actual fluid pressure, and addition of fluid to the system in the process of experiment or intermittent discharge of fluid from the system in the process of experiment, which can simulate episodic hydrocarbon discharge, that is, an intermittent discharge process of a simulated oil and gas caused by pressure changes underground. Gas, liquid hydrocarbon, water and solid residue produced in the pyrolysis experiment of the semi-closed system are collected and analyzed respectively, which can be used for related research.

However, after the pyrolysis experiment of the semi-closed system, all the products are placed open, where the solid residue is retained in a sample room of a heating furnace, and liquid product is retained in a solid-liquid separator, which are completely exposed to the air. The products continue to exposed to the air to undergo a heating separation process in subsequent routine experiments, therefore, liquid and solid products are subjected to obtain C₁₄⁺ non-volatile liquid hydrocarbons, while gas products are C₁-C₁₃ hydrocarbons. In subsequent analysis experiments of gas component and isotope composition under normal temperature and normal pressure, C₁-C₄ hydrocarbons are mainly analyzed. C₅⁺ hydrocarbons, which have low content, are volatile and have no special enrichment process, cannot obtain detection and analysis results, and are dispersed during the experiment.

When enrichment of the C₅⁺ hydrocarbons of the pyrolysis gas in the semi-closed system is carried out specifically, gas of C₁-C₄ hydrocarbons will be wasted, that is, two experiments are needed to realize a study of hydrocarbons with different components at the same time. In addition, the enrichment of C₅⁺ hydrocarbons from the C₁-C₁₃ hydrocarbons requires an introduction of other complicated devices and steps, which is time-consuming and laborious.

In addition, a content of C₅-C₁₃ light hydrocarbons in gas part of pyrolysis products of the semi-closed system is low (mostly between 10 ppm and 10³ ppm), and a content of their isotopes is below a normal detection limit of a mass spectrometer, which brings difficulties to analytical tests.

### SUMMARY

In order to overcome at least one defect of the prior art, in a first aspect, this application provides a semi-closed pyrolysis system capable of collecting C₅-C₈ light hydrocarbons.

In a second aspect, this application provides a method for determining a carbon isotope composition of light hydrocarbon monomers in a pyrolysis product of a semi-closed system.

Technical solutions of this application are described as follows.

In a first aspect, this application provides a semi-closed pyrolysis system capable of collecting C₅-C₈ light hydrocarbons, comprising:
a liquid nitrogen freezing device;
a gas-liquid collector; and
a pyrolysis gas collecting device;
wherein the liquid nitrogen freezing device is arranged between the gas-liquid collector and the pyrolysis gas collecting device;
the liquid nitrogen freezing device comprises a collecting vessel, a liquid nitrogen storage container containing liquid nitrogen, an air inlet pipe, an air outlet pipe and a temperature detector; the collecting vessel is arranged in the liquid nitrogen storage container; the collecting vessel is provided with a stainless steel cap; the stainless steel cap is provided with three holes; and the air inlet pipe, the air outlet pipe and the temperature detector are communicated with the collecting vessel through the three holes, respectively; the air inlet pipe is provided with an inlet valve, and the air outlet pipe is provided with an outlet valve; the temperature detector comprises a probe and a temperature sensor capable of displaying temperature readings; the probe is electrically connected with the temperature sensor; the probe is configured to extend into the collecting vessel; and the temperature sensor is arranged outside the collecting vessel; and
the air inlet pipe is connected with the gas-liquid collector; and the air outlet pipe is connected with the pyrolysis gas collecting device.

In an embodiment, the collecting vessel has a capacity of 10 ml, and is made of glass.

In an embodiment, a distance between a bottom of the collecting vessel and a bottom of the liquid nitrogen storage container is at least 2 cm.

In an embodiment, the pyrolysis gas collecting device comprises a water-discharging and gas-collecting device and a pyrolysis gas metering and storage device.

In an embodiment, the water-discharging and gas-collecting device comprises a first measuring cylinder, a second measuring cylinder and a funnel; a bottom of the first measuring cylinder is connected with a top of the second measuring cylinder; a bottom of the second measuring cylinder is connected with the funnel; the funnel comprises a spout and a funnel body; the funnel body has two openings, and the spout is connected with a smaller one of the two openings; and the funnel is open, and is directly communicated with an outside environment.

In an embodiment, the pyrolysis gas metering and storage device comprises a pyrolysis gas collecting bottle, a vessel containing a saturated NaCl aqueous solution and a pipe; the pyrolysis gas collecting bottle is arranged invertedly in the vessel; a first end of the pipe is inserted into the pyrolysis gas collecting bottle, and a second end of the pipe is connected with the first measuring cylinder; and a connection between the pipe and the first measuring cylinder is provided with a valve.

In an embodiment, the pyrolysis gas collecting bottle has a capacity more than 300 ml, and is made of glass.

In a second aspect, this application provides a method for determining a carbon isotope composition of light hydrocarbon monomers in a pyrolysis product of a semi-closed system, comprising:
(A) vacuumizing the semi-closed pyrolysis system; placing a hydrocarbon source rock sample which has a weight not less than 50 g and a diameter of 3-10 mm into the semi-closed pyrolysis system for a pyrolysis experiment; after the pyrolysis experiment is completed, filling the first measuring cylinder and the second measuring cylinder with a saturated NaCl aqueous solution, and closing a valve between the first measuring cylinder and a pipe of the pyrolysis gas metering and storage device and opening a hydrocarbon discharge valve of the gas-liquid collector;
adding liquid nitrogen into the liquid nitrogen storage container; opening a hydrocarbon discharge valve of the gas-liquid collector; successively opening the inlet valve and the outlet valve, so as to diffuse gas of the pyrolysis product into the collecting vessel with a capacity of 10 ml to enrich C₅-C₈ light hydrocarbons from the gas of the pyrolysis product;
manually adding the liquid nitrogen to maintain a liquid level of the liquid nitrogen at or above ⅔ of a height of the collecting vessel, and observing a reading of the temperature detector to maintain the reading within a range from −100° C. to −130° C.; after 60-90 s, completing collection of the C₅-C₈ light hydrocarbons, and then controlling the reading of the temperature detector to be less than or equal to −80° C.; unscrewing the collecting vessel from the stainless steel cap, and then sealing the collecting vessel with another cap to obtain a gas sample; and storing the collecting vessel at a temperature equal to or lower than −18° C. for analysis of a carbon isotope composition of C₅-C₈ light hydrocarbons; and
measuring, by the first measuring cylinder, remaining hydrocarbon gas in the pyrolysis gas collecting device; opening the valve between the first measuring cylinder and the pipe; and transferring the remaining hydrocarbon gas to a pyrolysis gas collecting bottle of the pyrolysis gas metering and storage device for a geochemical analysis of C₁-C₄ hydrocarbons in the pyrolysis product.

In an embodiment, in step (A), the hydrocarbon source rock sample is a rock core sample or a rock debris sample from shale, a marl or coal by drilling.

In an embodiment, in step (A), the pyrolysis gas collecting bottle is made of glass and has a capacity more than 300 ml.

In an embodiment, the distance between the bottom of the collecting vessel and the bottom of the liquid nitrogen storage container is at least 2 cm.

This application has the following beneficial effects.

This application applies a semi-closed system hydrocarbon generation pyrolysis experimental system which is most consistent with geological practice to perform pyrolysis of the hydrocarbon source rock sample. The semi-closed pyrolysis system is configured with the liquid nitrogen freezing device and adopts optimization method to maintain the liquid nitrogen at or above ⅔ of the height of the 10 ml collection bottle, so that the reading of the temperature detector is kept within a ranged from −100° C. to −130° C. After 60-90 s, the collection of the C₅-C₈ light hydrocarbons is completed, and C₁-C₄ light hydrocarbons with a capacity greater than 300 ml are also collected. The C₅-C₈ light hydrocarbons and C₁-C₄ light hydrocarbons can meet the needs of subsequent experiments.

Technical solutions of this application utilize the semi-closed system to carry out pyrolysis experiments on the hydrocarbon source rock sample. Besides “solid, liquid and gas (C₁-C₄ light hydrocarbons)” products which are conventionally obtained are not affected, C₅-C₈ light hydrocarbons which are in the “gas” are also enriched, which can enrich scientific researches on the geochemical characteristics of the pyrolysis products of the hydrocarbon source rock sample. The technical solutions of this application avoid introducing other complicated devices and steps to collect C₅-C₈ light hydrocarbons separately, and avoid carrying out additional repeated experiments (cost of each pyrolysis temperature point exceeds 1500 RMB). In addition, the technical solutions of this application save a consumption of precious hydrocarbon source rock sample samples.

### BRIEF DESCRIPTION OF THE DRAWINGS

FIG. 1 is a schematic diagram of a semi-closed high-temperature and high-pressure pyrolysis system (WYMN-3 high-temperature and high-pressure hydrocarbon generation simulator) according to an embodiment of the present disclosure.

FIG. 2 is a flow chart of a method for determining a carbon isotope composition of light hydrocarbon monomers in a pyrolysis product of a semi-closed system according to an embodiment of the present disclosure.

FIG. 3 shows a liquid nitrogen freezing device according to an embodiment of the present disclosure.

FIG. 4 shows a pyrolysis gas collecting device according to an embodiment of the present disclosure.

FIG. 5 is a spectrogram of a carbon isotope ratio of the light hydrocarbon monomers in a pyrolysis gas of a hydrocarbon source rock sample according to an embodiment of the present disclosure.

### DETAILED DESCRIPTION OF EMBODIMENTS

Unless otherwise defined, technologies and technical terms used herein have the same meaning as commonly understood by those of ordinary skill in the art. The technical terms used herein are only illustrative, and are not intended to limit this application.

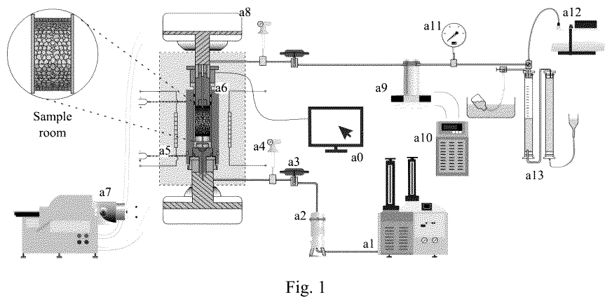

A semi-closed high-temperature and high-pressure pyrolysis system of the present disclosure is a WYMN-3 high-temperature and high-pressure hydrocarbon generation simulator, which is produced by Nantong Huaxing Petroleum Instrument Co., Ltd. FIG. 1 is a schematic diagram of a semi-closed pyrolysis system (in original factory condition), where in FIG. 1, a0 represents a computer system; a1 represents a high-pressure pump; a2 represents a middle vessel; a3 represents a pneumatic valve; a4 represents a pressure transmitter; a5 represents a thermocouple and heating furnace; a6 represents an autoclave body (internally provided with a sample room); a7 represents a hydraulic pump control system; a8 represents a hydraulic cylinder; a9 represents a gas-liquid collector (sleevedly provided with a cold trap); a10 represents a cooling water circulating machine; a11 represents a vacuum gauge; a12 represents a vacuum pump; and a13 represents a pyrolysis gas collecting device.

Components of a whole system are interconnected via stainless steel pipelines. The vacuum pump a12 is configured to interface with N pipes, where an air inlet of the vacuum pump a12 is connected to the N pipes in parallel, each one of the N pipes is equipped with a switch valve allowing selective operation, and the N pipes are designed to connected with any openings in a stainless steel pipeline network within the whole system. When specific location requires vacuum treatment, a corresponding switch valve of one of the N pipes connected to the specific location is opened, enabling vacuum application.

A working process of the WYMN-3 high-temperature and high-pressure hydrocarbon generation simulator is described as follows.

Sample preparation: An organic matter to be studied (such as coal, oil shale and sediment) is placed in the sample room (in the autoclave body a6).

Sealing of the sample room: The sealing of the sample room is ensured to prevent gas leakage during experiment.

Parameter setting: Parameters, such as a required temperature and a required pressure, are set through a control panel of the computer system a0, where the parameters are configured to simulate natural conditions underground.

Vacuum treatment: The N pipes of the whole system is subjected to the vacuum treatment.

Heating: A heating system, such as the thermocouple and heating furnace a5, is started, and the sample room is gradually heated to a set temperature.

Pressurization: The sample room of the autoclave body a6 is injected with ultrapure water through the high-pressure pump a1, the middle vessel a2, the pneumatic valve a3 and the pressure transmitter a4, to increase a fluid pressure in the system to a set value.

Monitoring: A temperature sensor (a first small probe set in the sample room of the autoclave body a6) is configured to monitor a temperature in the sample room in real time, and a pressure sensor (a second small probe set in the sample room of the autoclave body a6) is configured to monitor a pressure in the sample room in real time, so as to ensure a stability of the experimental conditions.

Data acquisition: The temperature, pressure and other possible parameters during the experiment are recorded by a data acquisition system of the computer system a0.

Reaction: At the set temperature and pressure, the organic matter in the sample is pyrolyzed and cracked to produce hydrocarbons. Hydrocarbon compounds are intermittently discharged and flow continuously into the gas-liquid collector a9. A gas-liquid mixture with high temperature and high pressure from the thermal simulator is separated in the gas-liquid collector a9, where a liquid component is remained at a bottom of the gas-liquid collector a9, and a gas component is at a top of the gas-liquid collector a9. The gas-liquid collector a9 is sleevedly provided with a metal heat conducting shell made of aluminum (that is, the cold trap).

Product collection: After the experiment, the thermocouple and heating furnace a5 is closed to reduce the temperature of the sample room. A hydrocarbon discharge valve of the gas-liquid collector a9 (separator) is opened, and the gas will be discharged. The hydrocarbons are collected through the pyrolysis gas collecting device a13.

Safety measures: in the process, a safety valve (set between the hydraulic cylinder a8 and the gas-liquid collector a9) is configured to ensure that the whole system can automatically release pressure when the pressure is too high, so as to protect safety of experimental personnel and equipment. The cooling water circulating machine a10 is configured to cool the whole system in the whole process.

End of the experiment: The heating system and a pressure system are turned off. The gas-liquid collector a9 is opened to obtain the liquid component. The sample room of the autoclave body a6 is opened to obtain the solid component for subsequent analysis.

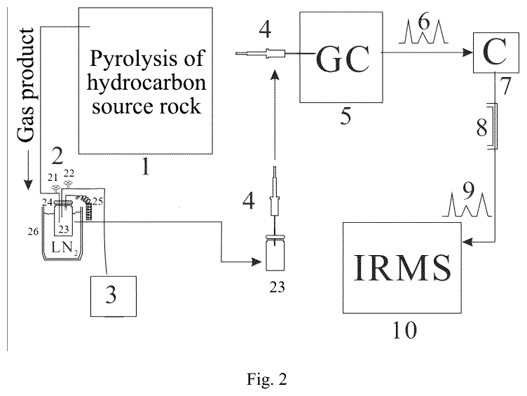

Referring to FIG. 2, the present disclosure provides the liquid nitrogen freezing device 2. The liquid nitrogen freezing device 2 is configured to enrich C₅-C₈ hydrocarbons in the pyrolysis gas. The liquid nitrogen freezing device 2 is arranged between the gas-liquid collector (a9 in FIG. 1) of the semi-closed pyrolysis system 1 and the pyrolysis gas collecting device 3 (a13 in FIG. 1).

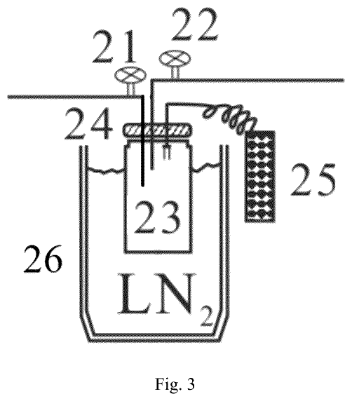

Referring to FIGS. 2-3, a semi-closed pyrolysis system capable of collecting C₅-C₈ light hydrocarbons is shown. The semi-closed pyrolysis system includes the liquid nitrogen freezing device 2, the gas-liquid collector and the pyrolysis gas collecting device. The liquid nitrogen freezing device 2 is arranged between the gas-liquid collector (a9 in FIG. 1) of the semi-closed pyrolysis system 1 and the pyrolysis gas collecting device 3.

The liquid nitrogen freezing device includes a collecting vessel 23 and a liquid nitrogen storage container 26. The collecting vessel 23 is set in the liquid nitrogen storage container 26. The collecting vessel 23 is provided with a stainless steel cap 24. The stainless steel cap 24 is provided with three holes, and the air inlet pipe, the air outlet pipe and the temperature detector are communicated the collecting vessel 23 through the three holes, respectively. The air inlet pipe is provided with an inlet valve 21, and the air outlet pipe is provided with an outlet valve 22. The temperature detector includes a probe and a temperature sensor capable of displaying temperature readings. The probe is electrically connected with the temperature sensor. The probe is configured to extend into the collecting vessel 23. The temperature sensor is arranged outside the collecting vessel 23.

The air inlet pipe is connected with the gas-liquid collector (a9 in FIG. 1) of the semi-closed pyrolysis system 1. The air outlet pipe of the liquid nitrogen freezing device 2 is connected with the pyrolysis gas collecting device 3.

Other components in FIG. 2 are: a solid-phase microextraction device 4, a gas chromatograph 5, a monomeric hydrocarbon compound 6, a combustion furnace 7, a water removal device 8, a dried CO₂ 9, an isotope ratio mass spectrometer 10 and the liquid nitrogen LN₂. A relevant experimental method is described as follows. A solid-phase microextraction head of the solid-phase microextraction device 4 in inserted in the collecting vessel 23 (gas sample) of the liquid nitrogen freezing device 2 for light hydrocarbon enrichment and extraction. Light hydrocarbon after the enrichment and extraction is separated through the gas chromatograph to obtain the monomeric hydrocarbon compound 6. The monomeric hydrocarbon compound 6 is transferred to the combustion furnace 7 to obtain a gas, and the gas is transferred to the water removal device 8 to obtain the dried CO₂ 9. The isotope ratio mass spectrometer 10 is configured to measure a carbon isotope composition in the monomeric hydrocarbon compound.

In an embodiment, the collecting vessel 23 has a capacity of 10 ml, and is made of glass.

In an embodiment, a distance between the collecting vessel 23 and a bottom of the liquid nitrogen storage container 26 is at least 2 cm. When such distance is less than 2 cm, the collecting vessel 23 is easy to stick directly to the bottom of the liquid nitrogen storage container 26, which is easy to cause bumps during the experimental operation, or it is easy to cause that the collecting vessel 23 is easy to stick to the bottom of the liquid nitrogen storage container 26 in a liquid nitrogen environment.

In an embodiment, an opening of the liquid nitrogen storage container 26 should not be too large, and is configured to just be put into a 10 ml collecting vessel and be convenient to add the liquid nitrogen. Owing to gasification of the liquid nitrogen at room temperature, the liquid nitrogen storage container 26 shall be provided with a slightly small opening.

In an embodiment, the liquid nitrogen storage container 26 has a capacity of 300-400 ml, and preferably 300-350 ml.

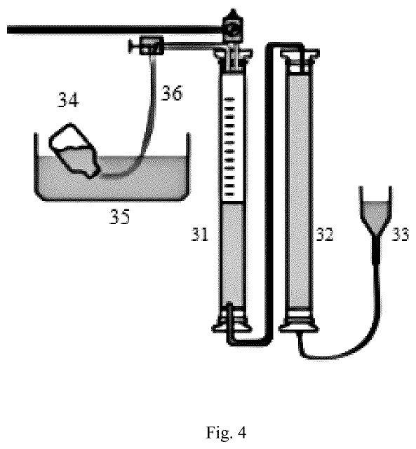

In an embodiment, referring to FIG. 4, the pyrolysis gas collecting device 3 includes a water-discharging and gas-collecting device and a pyrolysis gas metering and storage device.

In an embodiment, the water-discharging and gas-collecting device includes a first measuring cylinder 31, a second measuring cylinder 32 and a funnel 33, where a bottom of the first measuring cylinder 31 is connected with a top of the second measuring cylinder 32, and a bottom of the second measuring cylinder 32 is connected with the funnel 33. The funnel includes a spout and a funnel body, the funnel body has two openings, and the spout is connected with a smaller one of the two openings. The funnel is open and is directly communicated with an outside environment.

In an embodiment, the pyrolysis gas metering and storage device includes a pyrolysis gas collecting bottle 34, a vessel containing a saturated NaCl aqueous solution 35 and a pipe 36. The pyrolysis gas collecting bottle 34 is arranged invertedly in the vessel. A first end of the pipe 36 is inserted in the pyrolysis gas collecting bottle 34, and a second end of the pipe 36 is connected with the first measuring cylinder 31 of the water-discharging and gas-collecting device. A connection between the pipe 36 and the first measuring cylinder 31 is provided with a valve. When such valve is opened, the gas is diffused from the first measuring cylinder 31 of the water-discharging and gas-collecting device to the pyrolysis gas collecting bottle 34 through the pipe 36, and a saturated salt aqueous solution in the pyrolysis gas collecting bottle 34 is discharged, and gas in the pyrolysis gas collecting bottle 34 will gradually accumulate.

In an embodiment, the pyrolysis gas collecting bottle 34 has a capacity more than 300 ml, and is made of glass.

Collection of the pyrolysis gas is described as follows.

(S1) The semi-closed high-temperature and high-pressure pyrolysis system 1 (WYMN-3 high-temperature and high-pressure hydrocarbon generation simulator) is utilized to carry out the pyrolysis experiment. The hydrocarbon discharge valve of the gas-liquid collector a9 is always closed until the end of the experiment.

(S2) The valve between the pipe 36 and the first measuring cylinder 31 is closed, and the saturated salt aqueous solution is filled into the first measuring cylinder 31 and the second measuring cylinder 32 through the funnel 33 to completely remove air inside the first measuring cylinder 31 and the second measuring cylinder 32. Pipes behind the gas-liquid collector a9 of the semi-closed system thermal simulator are subjected to vacuum treatment. The collecting vessel 23 after vacuum treatment is placed, and the inlet valve 21 and the outlet valve 22 are examined and ensured to be closed.

(S3) The liquid nitrogen is poured into the liquid nitrogen storage container 26, so that a liquid level of the liquid nitrogen is maintained at or above ⅔ of a height of the collecting vessel 23, and the reading of the temperature detector is observed, so that the reading of the temperature detector is maintained within a range from −100° C. to −130° C.

(S4) The hydrocarbon discharge valve of the gas-liquid collector a9 of the semi-closed high-temperature and high-pressure pyrolysis system 1 is opened, at this time, the pyrolysis gas with high temperature and high pressure is diffused to where near the inlet valve 21 through the pipes.

(S5) The inlet valve 21 and the outlet valve 22 are successively opened. The pyrolysis gas with high temperature and high pressure is diffused through the inlet valve 21 to the collecting vessel 23 to fill the collecting vessel 23. In this process, C₅-C₈ hydrocarbons can be well enriched (frozen). Excess pyrolysis gas with high temperature and high pressure (mainly C₁-C₄ hydrocarbons) are discharged through the outlet valve 22, and then enter the first measuring cylinder 31 of the pyrolysis gas collecting device 3 through the pipes, and the saturated NaCl aqueous solution (SSS) in the first measuring cylinder 31 is discharged. A product collected in the collecting vessel 23 is represented as a gas sample, which mainly includes C₅-C₈ hydrocarbons, and inevitably includes a small amount of C₁-C₄ hydrocarbons in air state. A gas collected in the first measuring cylinder 31 is represented as a remaining hydrocarbon gas, which mainly includes C₁-C₄ hydrocarbons.

A content of C₉-C₁₃ hydrocarbons in the light hydrocarbons is generally very low, and is at least one or two orders of magnitude lower than that of C₅-C₈ hydrocarbons, which is below a detection line of the isotope ratio mass spectrometer. Therefore, C₉-C₁₃ hydrocarbons are not within the scope of the technical solutions of the present disclosure. In addition, a procedure of the isotope ratio mass spectrometer has an operation time of 25-32 min, and mainly collects C₅-C₈ hydrocarbons. Peaks of C₉-C₁₃ hydrocarbons with an extremely small amount generally appear after 35 min, therefore, C₉-C₁₃ hydrocarbons are excluded herein.

(S6) The reading of the temperature detector 25 is maintained within a range from −100° C. to −130° C. for 60-90 s. If the temperature drops quickly, the liquid nitrogen can be manually added.

Operation limited in Step (S6) is mainly related to boiling points of gaseous hydrocarbons, such as methane (a boiling point of methane is −162° C., a boiling point of ethane is −90° C., and the boiling points of gaseous hydrocarbons gradually increases as carbon number increases). The temperature of the 10 ml collecting vessel is controlled at −100° C.-−130° C., which can ensure that methane gas is maintained in the gas state, and will not be liquefied to an inner wall of the collecting vessel with C₅-C₈ hydrocarbons (a content of the methane is usually 90%-99.9% of hydrocarbon gas in the natural gas and the pyrolysis gas). The reading of the temperature detector 25 is maintained for 60-90 s, which is to allow C₅-C₈ light hydrocarbons to have sufficient time to liquefy and fix to the inner wall of the collecting vessel.

The reading of the temperature detector 25 is maintained for 60-90 s, so that gases of C₁-C₄ hydrocarbons, such as the methane, can be maintained in the gas state and will not be frozen by the liquid nitrogen. Continuously produced gases of C₁-C₄ hydrocarbons are discharged to the first measuring cylinder 31 through the outlet valve 22. Such time can obtain C₁-C₄ hydrocarbons nit less than 300 ml. It is inevitable that the 10 ml collecting vessel contains a small amount of C₁ methane gas, however, the methane accounts for a large proportion of a total gas, such loss of the methane has a small impact on subsequent results of values of C₁-C₄ carbon isotope, which is within an error range. In this way, a sufficient amount of C₅-C₈ hydrocarbons can be obtained in the 10 ml collecting vessel, and C₁-C₄ hydrocarbons with a volume more than 300 ml can also be obtained. C₁-C₄ hydrocarbons and C₅-C₈ hydrocarbons can meet the needs of the subsequent experiments well.

(S7) The inlet valve 21 and the outlet valve 22 are successively closed.

In step (S7), two valves are closed owing to time is enough. It can ensure that C₅-C₈ hydrocarbons is frozen, and discharged C₁-C₄ hydrocarbon gas can reach a requirement of the volume of 300 ml.

(S8) The reading of the temperature detector 25 is controlled to be less than or equal to −80° C. The collecting vessel 23 is quickly unscrewed from the (specially-made) stainless steel cap 24, and the collecting vessel 23 is sealed with a new cap with a rubber gasket. Then the collecting vessel 23 is subjected at a temperature equal to or lower than −18° C. Gas in the collecting vessel 23 is represented as the gas sample, which mainly includes C₅-C₈ hydrocarbons. Gas of C₅-C₈ hydrocarbons can be stored for up to 3 months in the environment with the temperature equal to or lower than −18° C. The C₅-C₈ hydrocarbons are used for analysis of the carbon isotope composition of the light hydrocarbons.

(S9) The gas sample obtained in step (S8) is properly stored at the temperature equal to or lower than −18° C., and then the remaining hydrocarbon gas obtained in the first measuring cylinder 31 of the water-discharging and gas-collecting device is transferred into the pyrolysis gas collecting bottle 34. A specific operation is described as follows. The pyrolysis gas collecting bottle 34 is arranged invertedly in the vessel containing saturated salt aqueous solution 35, so that an opening of the pyrolysis gas collecting bottle 34 is completely immersed in the saturated salt aqueous solution. The valve between the first measuring cylinder 31 and the pipe 36 is opened, and the liquid level in the funnel is slightly raised, and the remaining hydrocarbon gas in the first measuring cylinder is pressed into the 500 ml pyrolysis gas collecting bottle 34. The 500 ml pyrolysis gas collecting bottle 34 is sealed by a rubber plug and is kept upside down to prevent gas leakage, where the gas in the 500 ml pyrolysis gas collecting bottle 34 contains C₁-C₄ hydrocarbons, which is used for the conventional geochemical analysis of the pyrolysis gas.

A method for detecting a carbon isotope composition of light hydrocarbon monomers in a pyrolysis product of a semi-closed system includes the following steps.

(A) A hydrocarbon source rock sample which has a weight not less than 50 g and a diameter of 3-10 mm is placed in the semi-closed system thermal simulator 1 for a pyrolysis experiment. After the pyrolysis experiment, the pyrolysis gas is collected, where a collection method for the pyrolysis gas is described above.

(B) The gas sample stored at the temperature equal to or lower than −18° C. is subjected to solid-phase microextraction to achieve enrichment of hydrocarbons through the solid-phase microextraction device 4.

(C) The hydrocarbons are separated into the monomeric hydrocarbon compound 6 by the gas chromatograph 5.

(D) The monomeric hydrocarbon compound is analyzed to determine carbon isotope composition by the isotope ratio mass spectrometer 10.

The hydrocarbons are compounds composed of carbon and hydrogen atoms, mainly including alkanes, cycloalkanes, alkenes, alkynes and aromatic hydrocarbons.

The monomeric hydrocarbon compound mainly refers to single hydrocarbon compounds in petroleum fraction, especially gasoline fraction, such as normal alkanes, isomerized alkanes, alkenes, cycloalkanes and aromatic hydrocarbons.

A temperature of the liquid nitrogen is generally 77K (about −200° C.). The present disclosure provides the liquid nitrogen freezing device, so as to utilize the liquid nitrogen to freeze the collecting vessel (with the capacity of 10 ml and is made of glass) of the liquid nitrogen freezing device. Because the cooling of the liquid nitrogen is through the glass collecting vessel and is limited by factors, such as heat transfer efficiency and thermal performance of the glass bottle, the temperature in the collecting vessel can generally be stabilized within a range from −100° C. to −150° C.

It is researched that the temperature of the collecting vessel of the liquid nitrogen freezing device can be measured according to a height of the liquid nitrogen in the collecting vessel, where the higher the height of the liquid nitrogen, the lower the temperature in the collecting vessel. When the collecting vessel is completely filled with the liquid nitrogen, the temperature in the collecting vessel can reach a minimum of −150° C.; the lower the height of the liquid nitrogen, the higher the temperature in the collecting vessel.

The liquid nitrogen of the present disclosure can be manually replenished, so as to maintain the liquid level of the liquid nitrogen at the position of just ⅔ of the collecting vessel, which can maintain the temperature in the collecting vessel within a range from −100° C. to −130° C. In the temperature range from −100° C. to −130° C., the C₅-C₈ hydrocarbons are frozen well, where the word “frozen” herein refers to the C₅-C₈ hydrocarbons are turned into a liquid state at a low temperature, so that the C₅-C₈ hydrocarbons are adsorbed on the inner wall of the collecting vessel and are separated from the C₁-C₄ hydrocarbons, such as the methane (where the boiling points are too low to be frozen by the liquid nitrogen). When the liquid nitrogen in the liquid nitrogen storage container is too little, the cooling temperature in the collecting vessel cannot reach the requirement, and the C₅-C₈ hydrocarbons cannot be frozen. When the liquid nitrogen in the liquid nitrogen storage container is too much, the liquid nitrogen is easy to splash out, causing frostbite on an operator's hand skin, and affecting the stability of the experimental personnel when they screw the cap later.

In an embodiment, the gas sample (in the liquid state) in the collecting vessel 23 obtained in step (A) is transferred at the temperature equal to or lower than −18° C. and stored, which has the following objects.

At a low temperature of −18° C., the C₅-C₈ hydrocarbons will adhere in the liquid state to the inner wall or the bottom of the collecting vessel. In this way, C₅⁺ light hydrocarbons will not leak out slowly from the collecting vessel to the maximum extent. The gas sample obtained in the step (A) can be subjected to the following steps (B)-(D) immediately or subsequently according to the experimental arrangement.

Other hydrocarbon gas (remaining hydrocarbon gas) refers to the gas that is not frozen by the liquid nitrogen, mainly including gases of C₁-C₄ hydrocarbons, which is dominated by methane. Because of the boiling point of the methane is −161° C., the methane will not be frozen by the liquid nitrogen, and can be maintained in a gas phase. The methane will be subjected to output measuring through the first measuring cylinder 31 of the water-discharging and gas-collecting device, followed by transferring to the 500 ml pyrolysis gas collecting bottle 34.

In an embodiment, a chromatographic column used in the gas chromatograph 5 is selected from a HP-PONA column and a HP-AL/KCL column, which can retain C₅-C₈ hydrocarbons well.

In an embodiment, the chromatographic column of the gas chromatograph 5 is preferably HP-PONA column.

In an embodiment, the hydrocarbon source rock sample is a rock core sample or a rock debris sample from shale, marl or coal by drilling. The diameter of the hydrocarbon source rock sample is 3-10 mm.

In an embodiment, an amount of the of the pyrolysis gas (remaining hydrocarbon gas) collected in the pyrolysis gas collecting bottle is more than 300 ml.

Besides the C₅-C₈ hydrocarbons, subsequent use of conventional products obtained from the semi-closed system is described as follows.

(1) Solid residue is used to measure a maturity of hydrocarbon source rock sample (Ro), extract residual oil, analyze biological marker compounds and analyze carbon isotope composition of the solid residue.

(2) Liquid product (discharged oil) is used to analyze a total oil generation rate, analyze biological marker compounds of components in the discharged oil (saturated hydrocarbons, aromatic hydrocarbons, non-hydrocarbon, and asphaltene), analyze carbon isotope composition of a crude oil and the normal alkanes.

(3) Air product (C₁-C₄ hydrocarbons), that is, the remaining hydrocarbon gas collected in the pyrolysis gas collecting device 3, is used to measure a total gas production rate (for determining a gas generation potential of the hydrocarbon source rock sample), measure gas composition and measure carbon isotope composition of methane and ethane in the gas (for determining generation type and maturity of the pyrolysis gas).

Description above is a further detailed description of this application, which cannot be regarded as a limitation of a specific implementation of the present disclosure. For those of ordinary skill in the art, simple deductions and replacements made without departing the spirit of the present disclosure, shall fall within the scope of this application defined by the appended claims.

Experiment methods for which specific conditions are not indicated in the following embodiments of the present disclosure are usually in accordance with conventional conditions or conditions suggested by the manufacturer. Various commonly used chemical reagents used in the embodiments are commercially available products.

### EXAMPLE 1

A method for determining a carbon isotope composition of light hydrocarbon monomers in a pyrolysis product of a semi-closed system includes the following steps.

(S1) 300 g of a gray-black shale core sample with a total organic carbon content greater than 2% was obtained from a certain drilling well, followed by crushing into particles with diameters of 3.0-10.0 mm. 50.0 g of crushed gray-black shale core sample was placed into the sample room of the autoclave body a6, and the sample room was sealed through matching accessories including a retaining disc, a spacer, a copper ring, and a graphite gasket.

(S2) The sample room containing with the crushed gray-black shale core sample was arranged in the thermocouple and heating furnace a5 according to operating standard steps. According to pressure parameters of actual simulated formation, a hydraulic control system was utilized to correspondingly apply a lithostatic pressure (81.6 MPa), a surrounding rock pressure (137.0 MPa) and a fluid pressure (34.0 MPa).

(S3) The heating furnace was turned on, and maintained a heating rate of 7.5° C./h until the temperature of the heating furnace reached 350° C. and was kept for 72 h. During the heating and temperature-maintaining process, a preset procedure of the computer system a0 and an electronic control valve were utilized to perform hydrocarbon discharging and adjust fluid pressure in the system, so as to maintain the fluid pressure in the system in 30.6 MPa-40.8 MPa.

(S4) After 72 h temperature-maintaining process, the hydrocarbon discharge valve of the gas-liquid collector a9 of the semi-closed thermal simulator was opened, and the C₅-C₈ hydrocarbons in the gas product were enriched through the liquid nitrogen freezing device 2. After collection, the liquid nitrogen freezing device 2 was screwed and sealed through a new cap with polyethylene gasket, and then was immediately transferred to a freezer (less than −18° C.) for storage, which was prepared for subsequent analysis of the carbon isotope composition of C₅-C₈ hydrocarbons.

(S5) The remaining hydrocarbon gas, mainly including C₁ hydrocarbon (methane) was measured by the first measuring cylinder 31 of the water-discharging and gas-collecting device, followed by being collected in the 500 ml pyrolysis gas collecting bottle 34, and was seal with a rubber plug and kept upside down to prevent gas leakage for geochemical analysis of conventional pyrolysis gas. A carrier gas capacity of the pyrolysis gas collecting bottle 34 is 300 ml-350 ml.

(6) A syringe needle with a large size was used to pierce the rubber gasket at a center of the cap of the collecting vessel 23 in step (S4), so that when solid-phase microextraction head of the solid-phase microextraction device 4 passed through the rubber plug, it would not break an extracted fiber of the solid-phase microextraction head. A carbon molecular sieve/polydimethylsiloxane (CAR/PDMS) manual solid-phase microextraction head of the solid-phase microextraction device 4 which was activated at 300° C. for 3 h was inserted into the collecting vessel 23 for light hydrocarbon enrichment and extraction, where a balance time was 30 min, a temperature was 20° C. and a pressure was a standard atmosphere.

(S7) After the balance time, the solid-phase microextraction head of the solid-phase microextraction device 4 was removed from the collecting vessel 23, and then was immediately inserted into a sample inlet of the gas chromatograph 5, where a temperature of the sample inlet was 280° C. The solid-phase microextraction head of the solid-phase microextraction device 4 was kept in the sample inlet for 2 min, so that the light hydrocarbons adsorbed on the solid-phase microextraction head could be fully desorbed by pyrolysis.

(S8) The light hydrocarbons were subjected to chromatographic column separation through the gas chromatograph 5, the C₅-C₈ hydrocarbons in the light hydrocarbons were retained, and were separated into the monomeric hydrocarbon compound 6 (such as n-heptane, methylcyclohexane and toluene) through a multi-phase heating procedure.

(S9) After separation, the monomeric hydrocarbon compound 6 flowed from the gas chromatograph 5 to the combustion furnace 7 for oxidation-reduction reaction, and was transferred into CO₂ and H₂O.

(S10) CO₂ and H₂O obtained in the oxidation-reduction reaction in step (S9) passed through the water removal device 8 under the drive of carrier gas, so as to obtain corresponding CO₂ 9, and then the CO₂ 9 was introduced into the isotope ratio mass spectrometer 10.

(S11) The dried CO₂ 9 was ionized into three different isotope isomers with mass charge ratios (m/z) of 44, 45 and 46 respectively in an ion source of the isotope ratio mass spectrometer 10. Three charged ions with the three mass charge ratios (m/z) were separated through a magnetic field. A Faraday cup was used to receive signals, and carbon isotope ratios were calculated through the system.

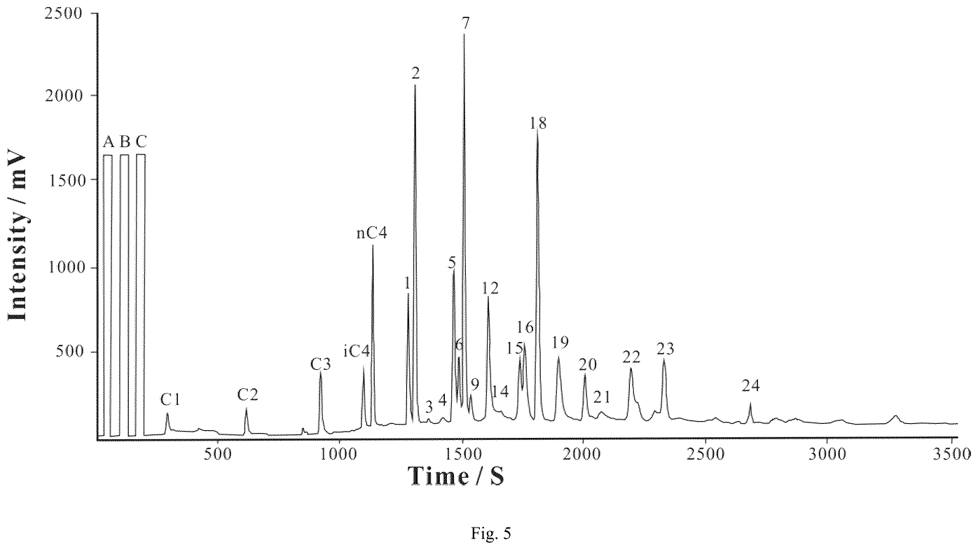

FIG. 5 is a spectrogram of a carbon isotope ratio of the light hydrocarbon monomers in the pyrolysis gas of the gray-black shale core sample under a preset heating temperature of 350° C. Carbon isotope composition characteristics of the light hydrocarbon monomers of the gray-black shale core sample are obtained through analysis of FIG. 5. Monomeric hydrocarbon compounds corresponding to peak numbers in FIG. 5 are: (1) isopentane; (2) n-pentane; (3) 2,2-dimethylbutane; (4) cyclopentane; (5) 2-methylpentane; (6) 3-methylpentane; (7) n-hexane; (8) 2,2-dimethylpentane; (9) methylcyclopentane; (10) 2,4-dimethylpentane; (11) 2,2,3-trimethylbutane; (12) benzene; (13) 3,3-dimethylpentane; (14) cyclohexane; (15) 2-methylhexane; (16) 2,3-dimethylpentane; (17) 1,1-dimethylcyclopentane; (18) 3-methylhexane; (19) 1,cis-3-dimethylcyclopentane; (20) 1,trans-3-dimethylcyclopentane; (21) 1,trans-2-dimethylcyclopentane; (22) n-heptane; (23) methylcyclohexane; and (24) methyl benzene.

The preset heating temperature in step (S3) in Example 1 was adjusted to 400° C., 450° C. and 500° C. respectively, and other reaction parameters were the same as those in Example 1, and Examples 2-4 were obtained.

Results of the carbon isotope composition of the light hydrocarbon monomers of the pyrolysis gas of the gray-black shale core sample under different preset heating temperatures (350° C.-500° C.) in Examples 1-4 are shown in Table 1.

TABLE 1

| | Temperature/ | δ¹³C(VPDB/%) | | | | | | | | | |
|---|---|---|---|---|---|---|---|---|---|---|---|
| Item | ° C. | 3-MC₅ | n-C₆ | Benz. | CC₆ | 3-MC₆ | n-C₇ | MCC₆ | Tol. | i-C₅ | n-C₅ |
| Example 1 | 350 | −31.2 | −33.6 | −27.2 | −26.4 | −32.1 | −30.3 | −29.1 | −26.3 | −30.5 | −28.4 |
| Example 2 | 400 | −31.7 | −30.9 | −28.5 | −29.6 | −32.1 | −30.8 | −28.6 | −26.8 | −31 | −29.1 |
| Example 3 | 450 | −30.3 | −30 | −28.7 | −29.3 | −30.4 | −30.3 | −27.1 | −26.9 | −30.2 | −31.1 |
| Example 4 | 500 | −31.1 | −32.1 | −26.6 | −27.8 | −31.3 | −30.9 | −28.4 | −26 | −29.9 | −30.4 |

Note:
δ¹³C(VPDB/%) represents the carbon isotope composition of corresponding compounds, where a standard is Vienna Peedee Belemnite (VPDB); where 3-MC₅ represents 3-methylpentane; n-C₆ represents n-hexane; Benz. represents benzene; CC₆ represents cyclohexane; 3-MC₆ represents 3-methylhexane; n-C₇ represents n-heptane; MCC₆ represents methylcyclohexane; Tol. represents methyl benzene; i-C₅ represents isopentane; and n-C₅ represents n-pentane.

If technical solutions were adjusted, all gases including C₁-C₄ hydrocarbons and C₅-C₈ hydrocarbons were directly collected by the pyrolysis gas collecting device, and C₅-C₈ hydrocarbons were enriched by solid phase microextraction (using HP-PONA column or HP-AL/KCL column) without using the light hydrocarbon enrichment device (as shown in FIGS. 2-3) and the hydrocarbons were separated into light hydrocarbon monomers by gas chromatography. In the process of determining the organic carbon isotope composition of many humic hydrocarbon source rocks (which tend to generate dry gas, that is, methane, and produce relatively few C₅⁺ light hydrocarbons during pyrolysis)) through the isotope ratio mass spectrometry, heights of peaks of many C₅⁺ compounds were too low to obtain accurate isotope values for individual hydrocarbons. As a result, heights of peaks, which were shown in FIG. 5, that are already relatively low, became even more pronouncedly low or even absent. Therefore, the technical solutions of the present disclosure can better enrich C₅-C₈ hydrocarbons.

In summary, utilizing the episodic hydrocarbon expulsion feature of the semi-closed system pyrolysis experiment device in this application to simulate the hydrocarbon generation process during in-situ mining of oil shale under actual geological conditions will yield more accurate results. In addition to retaining the original capabilities of the semi-closed system pyrolysis in obtaining liquid and solid residues, and part of products of the C₁-C₄ hydrocarbons, the C₅-C₈ hydrocarbons can also be obtained from the gas products. By comparing the C₅-C₈ hydrocarbons in the pyrolysis gas of the semi-closed system and the C₅-C₈ hydrocarbons in the natural gas, resulting data analysis and experiment results will be more intuitive and persuasive. The technology of the present disclosure can directly serve natural gas exploration, which has a great significance.

What is claimed is:

1. A semi-closed pyrolysis system capable of collecting C₅-C₈ light hydrocarbons, comprising:
a liquid nitrogen freezing device;
a gas-liquid collector; and
a pyrolysis gas collecting device;
wherein the liquid nitrogen freezing device is arranged between the gas-liquid collector and the pyrolysis gas collecting device;
the liquid nitrogen freezing device comprises a collecting vessel, a liquid nitrogen storage container containing liquid nitrogen, an air inlet pipe, an air outlet pipe and a temperature detector; the collecting vessel is arranged in the liquid nitrogen storage container; the collecting vessel is provided with a stainless steel cap; the stainless steel cap is provided with three holes; the air inlet pipe, the air outlet pipe and the temperature detector are communicated with the collecting vessel through the three holes, respectively; the air inlet pipe is provided with an inlet valve, and the air outlet pipe is provided with an outlet valve; the temperature detector comprises a probe and a temperature sensor capable of displaying temperature readings; the probe is electrically connected with the temperature sensor; the probe is configured to extend into the collecting vessel; and the temperature sensor is arranged outside the collecting vessel; and
the air inlet pipe is connected with the gas-liquid collector; and the air outlet pipe is connected with the pyrolysis gas collecting device.

2. The semi-closed pyrolysis system of claim 1, wherein the collecting vessel has a capacity of 10 mL, and is made of glass.

3. The semi-closed pyrolysis system of claim 1, wherein a distance between a bottom of the collecting vessel and a bottom of the liquid nitrogen storage container is at least 2 cm.

4. The semi-closed pyrolysis system of claim 1, wherein the pyrolysis gas collecting device comprises a water-discharging and gas-collecting device and a pyrolysis gas metering and storage device.

5. The semi-closed pyrolysis system of claim 4, wherein the water-discharging and gas-collecting device comprises a first measuring cylinder, a second measuring cylinder and a funnel; a bottom of the first measuring cylinder is connected with a top of the second measuring cylinder; a bottom of the second measuring cylinder is connected with the funnel; the funnel comprises a spout and a funnel body; the funnel body has two openings, and the spout is connected with a smaller one of the two openings; and the funnel is open, and is directly communicated with an outside environment.

6. The semi-closed pyrolysis system of claim 4, wherein the pyrolysis gas metering and storage device comprises a pyrolysis gas collecting bottle, a vessel containing a saturated NaCl aqueous solution and a pipe; the pyrolysis gas collecting bottle is arranged invertedly in the vessel; a first end of the pipe is inserted into the pyrolysis gas collecting bottle, and a second end of the pipe is connected with the first measuring cylinder; and a connection between the pipe and the first measuring cylinder is provided with a valve.

7. The semi-closed pyrolysis system of claim 6, wherein the pyrolysis gas collecting bottle has a capacity more than 300 mL, and is made of glass.

8. A method for determining a carbon isotope composition of light hydrocarbon monomers in a pyrolysis product of a semi-closed system, comprising:
(A) vacuumizing the semi-closed pyrolysis system of claim 5; placing a hydrocarbon source rock sample which has a weight not less than 50 g and a diameter of 3-10 mm into the semi-closed pyrolysis system for a pyrolysis experiment; after the pyrolysis experiment is completed, filling the first measuring cylinder and the second measuring cylinder with a saturated NaCl aqueous solution, and closing a valve between the first measuring cylinder and a pipe of the pyrolysis gas metering and storage device;
adding liquid nitrogen into the liquid nitrogen storage container; opening a hydrocarbon discharge valve of the gas-liquid collector; successively opening the inlet valve and the outlet valve of the liquid nitrogen freezing device, so as to diffuse gas of the pyrolysis product into the collecting vessel with a capacity of 10 mL to enrich C₅-C₈ light hydrocarbons from the gas of the pyrolysis product;
manually adding the liquid nitrogen to maintain a liquid level of the liquid nitrogen at or above ⅔ of a height of the collecting vessel, and observing a reading of the temperature detector to maintain the reading within a range from −100° C. to −130° C.; after 60-90 s, completing collection of the C₅-C₈ light hydrocarbons, and then controlling the reading of the temperature detector to be less than or equal to −80° C.; unscrewing the collecting vessel from the stainless steel cap, and then sealing the collecting vessel with another cap to obtain a gas sample; and storing the collecting vessel at a temperature equal to or lower than −18° C. for analysis of a carbon isotope composition of C₅-C₈ light hydrocarbons; and
measuring, by the first measuring cylinder, remaining hydrocarbon gas in the pyrolysis gas collecting device; opening the valve between the first measuring cylinder and the pipe; and transferring the remaining hydrocarbon gas to a pyrolysis gas collecting bottle of the pyrolysis gas metering and storage device for a geochemical analysis of C₁-C₄ hydrocarbons in the pyrolysis product;
(B) subjecting the gas sample stored at the temperature equal to or lower than −18° C. to solid-phase microextraction to achieve enrichment of C₅-C₈ light hydrocarbons;
(C) separating the C₅-C₈ light hydrocarbons into monomeric hydrocarbon compounds by gas chromatography; and
(D) analyzing the monomeric hydrocarbon compounds by using an isotope ratio mass spectrometer to determine carbon isotope composition of C₅-C₈ light hydrocarbon monomers, wherein an analysis time of the isotope ratio mass spectrometer is 26-32 min.

9. The method of claim 8, wherein in step (A), the hydrocarbon source rock sample is a rock core sample or a rock debris sample from shale, marl or coal by drilling.

10. The method of claim 8, wherein in step (C), a chromatographic column used in the gas chromatography is selected from a HP-PONA column and a HP-AL/KCL column.
